(19) United States
(12) Patent Application Publication
(10) Pub. No.: US 2015/0335827 A1
(43) Pub. Date: Nov. 26, 2015

(54) A Needle Device

(71) Applicant: NOVO NORDISK A/S, Bagsværd (DK)

(72) Inventors: Mads Schenstroem Stefansen, Copenhagen OE (DK); Henrik Bengtsson, Taastrup (DK)

(73) Assignee: NOVO NORDISK A/S, Bagsvaerd (DK)

(21) Appl. No.: 14/653,106

(22) PCT Filed: Dec. 16, 2013
(86) PCT No.: PCT/EP2013/076734

§ 371 Date: Jun. 17, 2015

Related U.S. Application Data
(60) Provisional application No. 61/739,878, filed on Dec. 20, 2012.

(30) Foreign Application Priority Data
Dec. 19, 2012 (EP) 12198082.5

(51) Int. Cl. A61M 5/32; A61M 5/24; A61M 5/34

(52) U.S. Cl. CPC A61M 5/322; A61M 5/3293; A61M 5/3295; A61M 5/343; A61M 5/2455; A61M 2202/0007; A61M 2005/3206

(57) ABSTRACT

The invention relates to a needle device which is made from a housing (1, 2, 3) having a distal wall (4) with a front (5). The housing further holds a compartment (21, 22) containing a liquid drug to be expelled. A needle cannula (10) for expelling the liquid drug having a distal end (1 1) and a proximal end (12) is also present, the needle cannula (10) being axially movable from a first position to a second position; The first position being a position in which the distal end (1 1) of the needle cannula (10) is retracted proximally behind the front (5) of the distal wall (4) of the housing (1, 2, 3), and wherein the proximal end (12) of the needle cannula (10) is located outside the first compartment (21, 22), and the second position being a position in which the distal end (1 1) of the needle cannula (10) is extended distally to the front (5) of the distal wall (4) of the housing (1, 2, 3), and wherein the proximal (12) end is located inside the compartment (21, 22).

THE TECHNICAL FIELD OF THE INVENTION

The invention relates to a needle device comprising one or more needle cannulae. The needle device is preferably attachable to an injection device for injecting a liquid drug. Alternatively the needle device can be part of an injection device.

### DESCRIPTION OF RELATED ART

A needle device comprising a plurality of needle cannulae is disclosed in US 2004/0162521. Each needle cannula is secured with the proximal end encapsulated in a needle carrier thus sealing off the proximal opening of each individual needle cannula. When moved to the injection position the individual needle cannula is penetrated through a chamber containing the drug to be injected. An opening in the sidewall of the needle cannula is thus brought into contact with the chamber as the distal part shoots forward beyond the distal front of the housing.

This needle device is however rather complex in its construction and at the same time it requires each individual needle to be provided with a side opening which complicate the manufacturing process.

### DESCRIPTION OF THE INVENTION

It is thus an object of the present invention to provide a needle device wherein traditional needle cannulae can be used without any extra and costly process steps. Further, it is an object to provide a needle device which can be attached to an injection device or alternatively be part of any well-known injection device including an injection pen.

The invention is defined in claim 1. Accordingly, in one aspect of the present invention, the needle device comprises a housing having a compartment and at least one needle cannula to be shifted axially between a first position and a second position.

The compartment can be an integral part of the housing or it can be a cartridge inserted into the housing, if being a cartridge it can either be permanently embedded in the housing or it can be exchangeable.

In the first position there is no liquid communication between the compartment and the lumen of the needle cannula as the proximal end of the needle cannula is located outside the compartment and inserted into a blocking element.

In the second position, the proximal end is moved axially out of the blocking element and into the compartment thereby creating a liquid communication between the compartment and the lumen of the needle cannula.

The housing of the needle device defines a distal front which preferably abuts against the skin of the user during injection. In the first position, the distal end of the needle cannula is retracted proximally behind the front of the distal front of the housing and in the second position the distal end of the needle cannula is extended distally out from the front of the housing into an injection position.

The core of the invention is that the needle cannula between injections is concealed in a retracted position. Once a user gets ready to inject he actively moves the needle cannula from the retracted position to an extended position. During this movement, the proximal end of the needle cannula moves from a position where the proximal opening of the lumen is shut off to a second position in which the proximal end is in liquid communication with the interior of the compartment containing the liquid drug. At the same time the distal end of the needle cannula is axially pulled or pushed from a position behind the front to the extended position wherein the distal end lies in front of the front.

Since the needle cannula is only pulled axially, a standard straight drawn needle cannula can be used, however it is not a requirement that the needle cannula is a straight needle cannula; it can alternatively be provided with bends. However, the distal end of the needle cannula is in both the first and in the second position located distally to the compartment containing the liquid drug.

The needle cannula is preferably any ordinary needle cannula having a distal pointed end with a first opening and an opposite located proximal end with a second opening. The first opening and the second opening is located on the same axis and a lumen extends axially connecting the first opening with the second opening. The needle cannula has preferably no other openings than the first and the second.

The blocking element sealing off the proximal opening of the needle cannula between injections is preferably an elastomeric component such as a rubber part. Which rubber part can be a part of the housing or alternatively a separate element provided inside the compartment.

When moving the needle cannula from the second position and proximally into the first position, a part of the rubber element will enter the proximal end of the lumen and squeeze the liquid content of the lumen out through the distal end of the needle cannula. Following this, the proximal end will be shut off i.e. the rubber part can be considered to be a valve arrangement shutting of flow through the lumen of the needle cannula.

The liquid communication between the interior of the compartment containing the liquid drug and the lumen of the needle cannula can be executed through another chamber. This chamber can e.g. be formed as a pocket which is in liquid communication with the remaining compartment. This pocket can either be an integral part of the compartment or it can be connected to the compartment e.g. via a conduit. In one example, the pocket can be formed in a needle assembly and the remaining compartment can be formed in the injection device. In a different example, both the compartment and the pocket can be integral parts of a cartridge.

The proximal end of the needle cannula is preferably shifted axially between a sealed position wherein the proximal end is maintained proximal to the pocket to a position wherein the proximal end of the needle cannula is inside the pocket thus creating a liquid flow.

The liquid communication between the pocket and the remaining compartment can be established via a flow channel e.g. provided in the rubber part sealing off the proximal end of the needle cannula between injections.

The needle cannula is preferably connected with a slidable element by which the user can shift the needle cannula axially between the first and the second position. This slider could preferably be connected to the needle cannula via a hub. The needle cannula is thus attached to the hub which is engaged by the slider.

Further, the needle device, which can be provided with any number of needle cannulae, can be formed integrally with an injection device or it can be a separate needle assembly which is attachable to an injection device. In the latter case, the needle assembly preferably contains only one single needle cannula.

### DEFINITIONS

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

The term “Needle Cannula” is used to describe the actual conduit performing the penetration of the skin during injection. A needle cannula is usually made from a metallic material such as e.g. stainless steel and connected to a hub to form a complete injection needle also often referred to as a “needle assembly”. The needle cannula could however also be made from a polymeric material or a glass material. A needle assembly especially designed for use with an injection pen is defined in ISO standard No. 11608, part 2, and are often referred to as “pen needles”. Pen needles or pen needle assemblies have a front-end for penetrating into the user and a back-end for penetrating into the cartridge containing the liquid drug.

“Cartridge” is the term used to describe the container containing the drug. Cartridges are usually made from glass but could also be moulded from any suitable polymer. A cartridge or ampoule is preferably sealed at one end by a pierceable membrane referred to as the “septum” which can be pierced e.g. by the back-end of a needle cannula. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1A show a perspective view of the needle device.

FIG. 1B show a cross sectional view of the needle device.

FIG. 2A-B show an embodiment of the needle device attached to an injection device.

FIG. 2C show a housing part of FIG. 2A-B

FIG. 3A-B show another embodiment of the needle device attached to an injection device.

FIG. 4A-B show yet a different embodiment of the needle device wherein the compartment and the pocket thereof is provided in the cartridge.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

In that context it may be convenient to define that the term “distal end” in the appended figures is meant to refer to the end of the needle cannula which penetrates into the users skin whereas the term “proximal end” is meant to refer to the opposite end of the needle cannula which enters into the container containing the liquid drug.

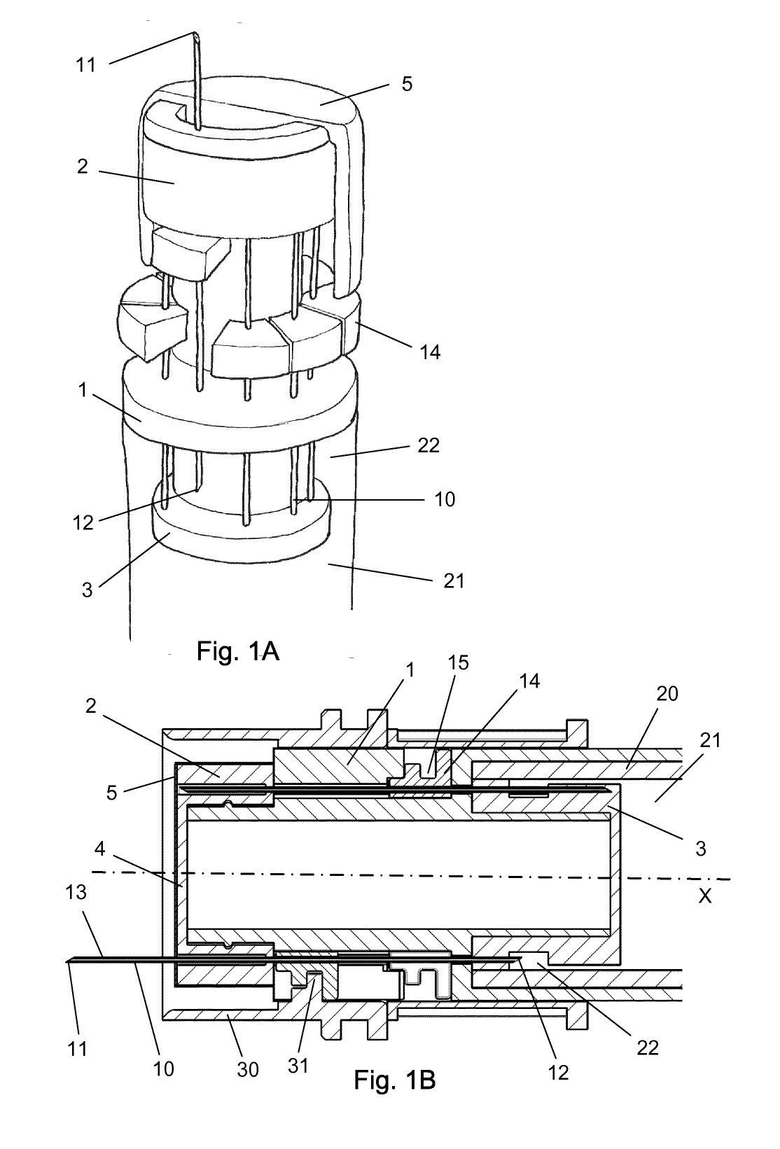

FIG. 1A-B discloses the needle device of the invention comprising a housing 1, 2, 3. The housing can be manufactured as a unitary part or it can, as in FIG. 1A-B, be made from multiple housing parts 1, 2, 3 that are glued, welded or otherwise connected together.

At the distal end, the housing 1, 2, 3 is provided with a distal wall 4 having a front 5 from which the individual needle cannulae 10 can extend during injection.

Each needle cannula 10 has a distal end 11, a proximal end 12 and a lumen 13 extending there between. The openings of the distal end 11 and of the proximal end 12 is preferably provided on the same axially extending axis, preferably running parallel to the centre axis X, as it is known from traditional injection needles. Further each individual needle cannula 10 is secured in a hub 14

The housing parts 1, 2, 3 seals off a cartridge 20 containing a liquid drug in a hollow compartment 21. This compartment 21 distally forms a pocket 22 which also holds the liquid drug contained in the compartment 21. The interior of this pocket 22 is in liquid communication with the compartment 21 of the cartridge 20.

A slidable guide 30 can be provided on the outside of the housing parts 1, 2, 3 which guide 30 can be manually slided axially by a user. The slidable guide 30 can be provided with a protrusion 31 engaging a groove 15 in the hub 14 in which the individual needle cannula 10 is secured.

By sliding the slidable guide 30 the individual needle cannula 10 can be moved from a first position to a second position. In between the axial movement of the slidable guide 30 it can be rotated to engage the subsequent needle cannula 10 such that the following needle cannula 10 can be used for the subsequent injection.

The first position is shown on the upper part of FIG. 1 B and is the initial position for the needle device. In this position, the distal end 11 of the individual needle cannula 10 is retracted proximally behind the front 5 of the distal wall 4 of the housing 1, 2, 3, and the proximal end 12 of the individual needle cannula 10 is located outside the compartment 21 i.e. there is no liquid communication between the lumen 13 and the compartment 21.

As can be best seen in FIG. 1B, the proximal end 12 of the individual needle cannula 10 is embedded in a housing part 3 which is preferably made from rubber or the like.

The second position is shown in the lower part of FIG. 1B. In this position, the distal end 11 of the individual needle cannula 10 is extended beyond the front 5 of the wall 4 of the housing 1, 2, 3 and the proximal end 12 has been moved into the pocket 22 of the chamber or compartment 21 such that liquid communication between the compartment 21 and the distal end 11 of the individual needle cannula 10 is established.

Prior to an injection, the user slides the slidable guide 30 in the distal direction which thrust the distal end 11 of one needle cannula 10 forward while moving the proximal end 12 of the same needle cannula 10 into liquid communication with the liquid drug in the pocket 22 of the compartment 21 thus making the device ready to inject.

The slidable guide 30 can be rotational mounted on the housing parts 1, 2, 3 such that it can be shifted from one needle cannula 10 to the next consecutive needle cannula 10.

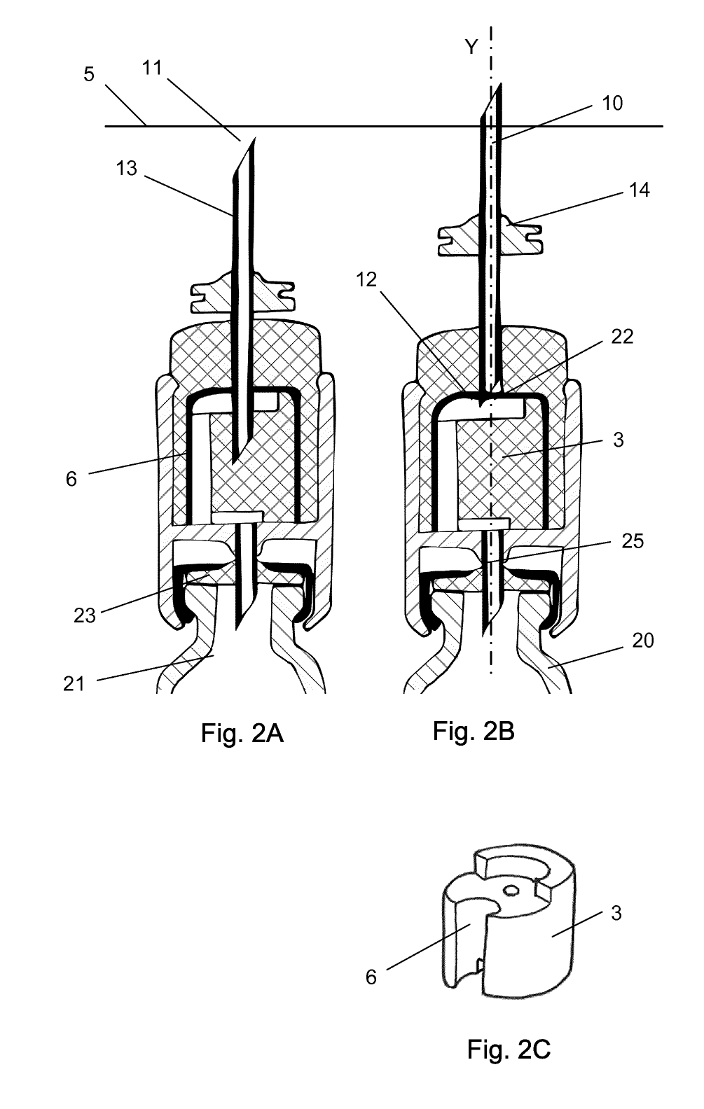

An embodiment having only one needle cannula 10 is disclosed in FIG. 2A-B. In this embodiment, the cartridge 20 has a chamber or compartment 21 holding the liquid drug, which compartment 21 is connected to the pocket 22 via a conduit 25. The conduit 25 penetrates through the septum 23 of the cartridge 20. The cartridge 20 and its compartment 21 can in one embodiment be part of an injection device whereas the pocket 22 can be part of a needle assembly which is attachable to the injection device. The housing part 3 (FIG. 2C) in which the proximal end 12 of the needle cannula 10 is inserted in the initial—first—position is preferably made from a rubber material and has a channel 6 through which the liquid drug can flow from the conduit 25 and into the pocket 22. The rubber element 3 functions as a valve shutting off the liquid communication between the pocket 21 (being part of the compartment 21) and the lumen 13 of the needle cannula 10.

In FIG. 2A, the distal end 11 of the needle cannula 10 is in its retracted position hidden behind the front 5, and the proximal end 12 is embedded in the housing part 3. In FIG. 2B, the distal end 11 has been moved to its extended position in which the distal end 11 of the needle cannula 10 extends beyond the front 5 and the proximal end 12 is in liquid communication with the pocket 22 which again is in liquid communication with the compartment 21 of the container 20.

The axial movement of the needle cannula 10 is preferably done by axially moving the hub 14 to which the needle cannula 10 is attached.

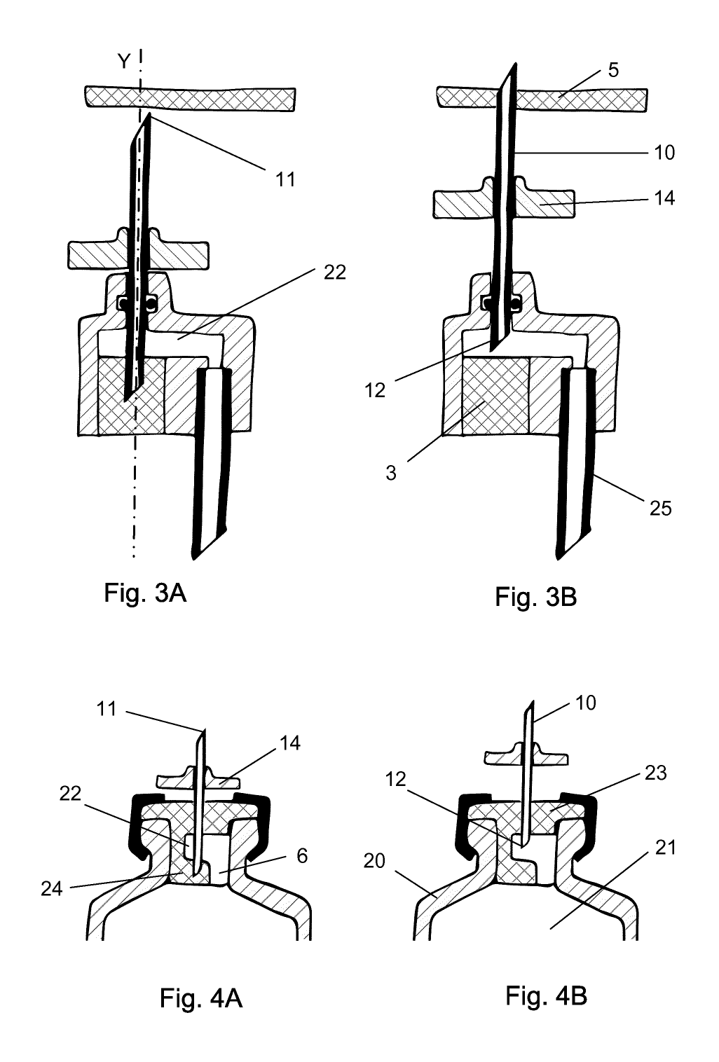

Also in the embodiment depictured in FIG. 3A-B, the hub 14 is moved axially to thereby move the needle cannula 10 between its two end positions.

In the embodiment of FIG. 3A-B, the conduit 25 is provided dislocated in relation to the center axis Y of the needle cannula 10.

The initial position with the distal end 11 of the needle cannula 10 in the retracted position and the proximal end 12 encapsulated inside the housing part 3 is disclosed in FIG. 3A.

In FIG. 3B the system (the injection device with the needle assembly mounted thereon) is in the “Ready-to-Inject” position. The distal end 11 is extended beyond the front 5, and the proximal end 12 is in liquid communication with the compartment 22 which again is in liquid communication with the compartment 21 of the container 20 through the tube 25.

FIG. 4A-B discloses an embodiment in which the pocket 22 is formed integral with the septum 23 of the cartridge 20. As can be seen, the neck portion of the cartridge 20 is sealed off by the septum 23 which has a rubber extension 24 which together with a channel 6 defines the pocket 22.

The needle cannula 10 anchored in the hub 14 is attached by the user. The needle cannula 10 could be one of an ordinary pen needle assembly. The pen needle assembly is preferably attached as shown in FIG. 4A, whenever the user needs to perform an injection, the pen needle assembly with the needle cannula 10 only have to pulled forward a very short distance such that the proximal end 12 of the needle cannula 10 is moved into the pocket 22 as shown in FIG. 4B. Once the injection has been performed, the user can push the pen needle assembly with the needle cannula 10 proximately to shut off the valve mechanism i.e. move the proximal part 12 of the needle cannula 10 into the extension 24 loctade proximately on the septum 23.

Some preferred embodiments have been shown in the foregoing, but it should be stressed that the invention is not limited to these, but may be embodied in other ways within the subject matter defined in the following claims.

1. A needle device comprising:
a housing having a distal wall with a front,
a compartment containing a liquid drug to be expelled, and
a needle cannula for expelling the liquid drug having a distal end and a proximal end and a lumen extending there between, the needle cannula being axially movable relatively to the compartment from a first position to a second position;
the first position being a position in which the proximal end of the needle cannula is located outside the compartment and embedded in a blocking element thereby preventing liquid communication between the compartment and the lumen whereas the distal end of the needle cannula is retracted proximally behind the front of the distal wall, and
the second position being a position in which the proximal end is located inside the compartment thereby allowing liquid communication between the compartment and the lumen whereas the distal end of the needle cannula is extended distally to the front of the distal wall of the housing.

2. A needle device according to claim 1, wherein the distal end has a first opening and the proximal end has a second opening, the first opening and the second opening being located on the same axis and wherein the lumen extend axially connecting the first opening with the second opening.

3. A needle device according to claim 1, wherein the blocking element is an elastomeric component.

4. A needle device according to claim 3, wherein the elastomeric component is provided in the reservoir.

5. A needle device according to claim 1, wherein the compartment comprises a pocket in liquid communication with the remaining compartment.

6. A needle device according to claim 5, wherein the elastomeric component is provided with a flow channel connecting the pocket and the remaining reservoir.

7. A needle device according to claim 5, wherein the proximal end of the needle cannula in the second position is located inside the pocket.

8. A needle device according to claim 1, wherein the proximal end of the needle cannula in the first position is located proximally to the pocket.

9. A needle device according to claim 1, wherein a slider is provided for shifting the needle cannula between the first position and the second position.

10. A needle device according to claim 1, wherein the needle cannula is attached to a hub.

11. A needle device according to claim 10, wherein the slider engages the hub.

12. A needle device according to claim 1, wherein needle device is attachable to an injection device.

13. A needle device according to claim 1, wherein needle device carries a plurality of needle cannulae.
